(19) United States
(12) Patent Application Publication
(10) Pub. No.: US 2004/0072258 A1
(43) Pub. Date: Apr. 15, 2004

(54) Isoprenoid-dependent ras anchorage (idra) proteins

(76) Inventors: Yoel Kloog, Herzlia (IL); Roni Haklai, Ramat Gan (IL); Ariella Paz, Kfar-Saba (IL); Galit El Ad-Sfadia, Raanana (IL); Eyal Ballan, Hod Hasharon (IL)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/398,519

(22) PCT Filed: Oct. 1, 2001
(86) PCT No.: PCT/IL01/00918

(51) Int. Cl. G01N 33/53; G01N 33/567; A61K 31/4415; A61K 31/195

(52) U.S. Cl. 435/7.2; 514/557; 514/350; 514/567

(57) ABSTRACT

Disclosed is the identity of various Ras cell membrane anchor proteins. Also disclosed are methods for identifying other anchor proteins that bind isoforms of Ras, methods of identifying drug candidates that inhibit aberrant Ras activity and methods of determining therapeutic dosages of the drugs. Further disclosed are methods for disrupting aberrant Ras activity in vivo.

### TECHNICAL FIELD

The present invention relates to Ras proteins, and more specifically to interactions between Ras and other cellular proteins.

### BACKGROUND OF THE INVENTION

Ras proteins must be anchored to the inner surface of the cell membrane to function as cellular regulators of the signal transduction pathways controlling cell growth, differentiation, survival, and transformation [Kloog et al., 1999]. Membrane anchorage of Ras proteins is promoted by their C-terminal S-farnesylcysteine, by a stretch of lysines in K-Ras 4B, or by the S-farnesylcysteine and S-palmitoyl moieties in H— and N-Ras, suggesting the concept of a two-signal mechanism for Ras membrane targeting and association [Casey et al., 1989; Hancock et al., 1989; Cox et al, 1992]. In addition, intact sequences around the palmitoylation site are also required for proper targeting, indicating a three-signal mechanism [Willumsen et al., 1996].

The anchoring moieties of Ras proteins appear to target them to the plasma membrane [Cox and Der, 1997] and possibly to specific microdomains [Song et al., 1996; Mineo et al., 1996; Engelman et al., 1997; Mineo et al., 1997]. The mechanism of the farnesyl-dependent Ras membrane anchorage remains unknown. However, several experiments suggest that the farnesyl moiety common to all Ras proteins serves as a lipophilic lipid anchor and, in addition, confers functional specificity on Ras. For example, H-Ras modified by an inappropriate isoprenoid (e.g., by the C₂₀ geranylgeranyl group) has transforming activity but not normal Ras function [Buss et al., 1989]. Other experiments showed that modification of inactive unfarnesylated normal Ras by the fatty acid myristate results in activation of transforming activity, thus suggesting that myristoylated Ras (myr-Ras) cannot control normal Ras functions [Buss et al., 1989]. Furthermore, measurements of the association constants for Ras model peptides, modified by various lipids, to lipid vesicles showed that farnesylated peptides bind with a relatively low affinity [Shahinian and Silvius, 1995; Schroeder et al., 1997]. These studies suggest that the branched side-chain structure of farnesyl is an inferior lipid glue when compared to other lipids such as myristate or palmitate [Gelb et al., 1998]. Several lines of evidence are consistent with the notions that Ras is not glued nonspecifically to the cell membrane but rather is selectively tethered in specific membrane microdomains, possibly associated with specific receptors or anchors [Siddiqui et al., 1998], and that interactions of Ras with such domains are dynamic in nature [Niv et al., 1999].

The experiments reviewed above raise the possibility that the farnesyl group, common to all Ras proteins, acts as part of a recognition unit for specific anchorage lipids or protein(s) Ras in the cell membrane [Cox and Der, 1997]. On the assumption that Ras functions would be inhibited by competitive displacement of the mature protein from its putative membrane-anchorage domains, a series of organic compounds resembling the farnesylcysteine of Ras proteins were designed [Marciano et al., 1995; Marciano et al., 1997; Aharonson et al., 1998]. Among these compounds, S-trans, trans-farnesylthiosalicylic acid (FTS), was found to be a potent growth inhibitor of H-Ras-transformed Rat-I (EJ) fibroblasts. This compound and several of its active analogs were effective in a concentration range of 5-50 [symbol] M and affected specifically the membrane-bound H-Ras protein in these cells [Marciano et al., 1995; Marom et al., 1995; Aharonson et al., 1998; Haklai et al., 1998]. The observed stringent structural requirement for anti-Ras activity among S-prenyl analogs suggested specific protein binding [Aharonson et al., 1998]. Significantly, FTS and its C₂₀ geranylgeranyl analogue (GGTS), but not its Clo geranyl analogue (GTS) or its carboxy methylester analogue, inhibited growth of Ras-transformed cells [Aharonson et al., 1998]. The demonstration that FTS inhibits the growth of fibroblasts transformed by ErbB2 acting upstream of Ras, but not the growth of cells transformed by v-Raf, which unlike Raf-1 acts independently of Ras, suggested specificity of FTS towards Ras [Marom et al., 1995]. Mechanism of action studies showed that FTS did not inhibit farnesylation of H-Ras [Marom et al., 1995]. It affected H-Ras membrane interactions in intact cells in vitro by dislodging the protein from its anchorage domains, which facilitated its degradation and thus reduced the total amount of cellular Ras [Haklai et al., 1998]. Although FTS and other S-prenyl analogues inhibit Ras methylation in vitro [Marciano et al., 1995; Aharonson et al., 1998], its growth-inhibiting effects in intact cells occur at concentrations lower than those required for inhibition of methylation [Marom et al., 1995]. Additional studies showed that FTS inhibits growth of cells transformed with the farnesylated but unmethylated K-Ras 4B (12V) CVYL isoform, confirming that methylation of Ras is not necessary for transformation [Elad, et al., 1999]. Further studies demonstrated that FTS also dislodges the normally processed K-Ras 4B (12V), N-Ras (13V) and N-Ras (61L) isoforms from membranes of rodent fibroblasts [Elad, et al. 1999; Jansen et al., 1999] and from membranes of human tumor cell lines [Jansen et al., 1999; Weisz et al., 1999; Egozi et al., 1999]. The effects of FTS appeared to be specific to the Ras protein. For example, FTS did not dislodge the prenylated Gβγ-subunits of heterotriimeric G-proteins from Rat-1 cell membranes and had no effect on myr-Ras in myr-Ras-transformed cells [Haklai et al., 1998]. Recent studies also showed that FTS did not reduce the amounts of prenylated Rac-1 and Rho A in human melanoma cells [Jansen et al., 1999].

### SUMMARY OF THE INVENTION

Applicants have isolated Ras-interacting proteins termed IDRA (isoprenoid-dependent Ras anchorage proteins). Ras-IDRA protein complexes were identified in extracts of membranes from H-Ras (12V)-transformed Rat-1 (EJ) cells. IDRAs were isolated from such complexes and identified by MS and by specific antibodies as galectin-1, a mammalian protein associated with cell growth and transformation [Perillo et al., 1998]. On the basis of in vivo and in vitro studies, Applicants have established that this is an anchor protein for Ras, and particularly the H-Ras isoform. In similar experiments, galectin-3 was identified as an anchor protein for the K-Ras isoform; galectin-7 and galectin-8 were identified as anchor proteins for multiple Ras isoforms.

One aspect of the present invention is directed to a method for identifying a cell membrane anchor protein that binds an isoform of Ras. The method entails preparing two reaction mixtures containing a source of a Ras protein and its anchor protein, e.g., intact cells, cell lysate, cell membranes or fractions thereof. One reaction mixture also contains a Ras antagonist. A cross-linking agent is added to both reaction mixtures whereby cross-linked complexes between Ras and cell membrane proteins are produced. The cross-linked complexes formed in both reaction mixtures are separated individually. The Ras-protein complex (formed in the reaction mixture without the antagonist) that is disrupted by the Ras antagonist (present in the other reaction mixture) is identified. That complex is isolated from the other complexes, and then the Ras protein is separated from the other protein(s) in that complex. Preferred Ras antagonists are FTS and analogs thereof. In other preferred embodiments, the individual separation of the cross-linked complexes and identification of the complex disrupted by antagonist are conducted by fractionating the two reaction mixtures side-by-side on a gel. The method may be used to identify anchor proteins for prenylated isoforms of Ras such H-Ras, K-Ras4A, K-Ras4B and N-Ras, generally regarded as the “classic” Ras isoforms, as well as for prenylated Ras regulatory proteins such as Rac and Rho, and non-prenylated Ras regulatory proteins such as Rit and Rin. For purposes of the present invention, all such Ras proteins are referred to interchangeably as Ras isoforms or Ras proteins.

Another aspect of the present invention is directed to a method for identifying drug candidates that inhibit aberrant Ras activity. This method entails preparing living cells or a reaction mixture containing a Ras protein, one or more anchor proteins that bind the Ras protein and the drug candidate, and determining the effect of the drug candidate on the interaction of Ras and the anchor protein. The effect of the drug on Ras-anchor interaction can be measured in a variety of ways. In one embodiment, the change in the extent of dimerization of the Ras isoform is measured. In another embodiment, the change in the extent of binding or activation of Raf protein is determined. In yet another embodiment, the change in the extent of binding between the Ras isoform and the anchor protein is measured in a reaction mixture or an intact cell. In preferred embodiments, the Ras isoform or the anchor protein is immobilized on a matrix. In other preferred embodiments, the anchor protein is galectin-1, galectin-3, galectin-7 or galectin-8.

Further aspects of the present invention are directed to a method for disrupting aberrant Ras activity in vivo, and compositions for use therewith. The method entails administering to a patient exhibiting aberrant Ras activity specific oligonucleotides that bind and inactivate the mRNA of an anchor protein for Ras. The specific oligonucleotides bind mRNA of the anchor protein and thus decrease its expression that in turn decreases Ras activity. In preferred embodiments, the specific oligonucleotides bind galectin-1, galectin-3, galectin-7 or galectin-8 mRNA. Combinations of antisense oligonucleotides that bind different of these proteins are also contemplated. The compositions contain an oligonucleotide that specifically targets a nucleic acid encoding a Ras anchor protein which binds (e.g., hybridizes) the nucleic acid and causes degradation of the nucleic acid. Preferred antisense oligos bind nucleic acids encoding galectin-1, galectin-3, galectin-7 or galectin-8.

Yet another aspect of the present invention is directed to a method of determining efficacious dosages of a Ras antagonist that disrupts Ras-anchor protein binding, comprising:

(i) contacting cells with the antagonist in vivo or in vitro;

(ii) collecting the cells following said contacting;

(iii) isolating cell membranes from the collected cells;

(iv) measuring decrease in anchor protein concentration per unit of cell membrane protein; and

(e) correlating the decrease with dosage of the Ras antagonist.

### BRIEF DESCRIPTION OF THE DRAWINGS

FIG. 1 is a photograph of an electrophoretic gel illustrating use of Ras antibodies and chemical cross-linkers for identification of Ras IDRA complexes that are sensitive to the Ras inhibitor, FTS. Ras antibodies (Ab) identify Ras and Ras-IDRA complex in EJ cell membranes. Membranes corresponding to 10⁶ control or FTS (50 μM)-treated EJ cells were incubated in 50 mM sodium bicarbonate buffer, pH 8.5, containing protease inhibitors and 2% DMSO (no cross-linker controls) or the indicated concentrations of the cross-linkers DSS or DSP. Samples of Triton X-100 extracts of the membranes were subjected to SDS-PAGE under non-reducing conditions followed by Western immunoblotting with Ras Ab. Open arrow corresponds to Ras (21 kDa) and closed arrow corresponds to the major Ras-putative IDRA band (34-43 kDa) detected in the blot under these conditions. This band is not detected in the blots of the non cross-linked samples or in the blots of the cross-linked samples of FTS-treated cells.

FIG. 2 is a photograph of a Western blot of galectin-1 isolated from cells with (+) and without (−) FTS treatment. FTS blocks the interaction of Ras with its anchor, and reduces the amounts of anchor in treated cells.

FIG. 3 is a photograph of a Western blot of galectin-1 isolated from cells in the presence of a control, FTS and GTS. FTS reduced the amount of membrane-associated galectin-1 by 90% while the inactive analog of FTS (GTS) had no effect.

FIG. 4 contains photographs of electrophoretic gels. Left Panel: membranes from five cell types, each with a different Ras isoform, were cross-linked, extracted, and fractionated on gels as in FIG. 1 (EJ and Rat1 cells that contain oncogenic and wild-type H-Ras respectively; myr-Ras does not bind to any anchor protein). The 34-43 kDa band was identified with Ras antibody, extracted, and run on a second gel. Right Panel: The Ras proteins, galectin-1 (Gal-1) and galectin-3 (Gal-3), released under reducing conditions from the 34-43 kDa cross-linked proteins form membranes isolated from cells transformed with various oncogenic Ras isoforms.

FIG. 5 is a photograph of a Western blot, illustrating that antisense Gal-1 reduces the expression of H-Ras (12V). C7-7 or 293T cells transfected with oncogenic H-Ras in the vector pcDNA3 resulted in expression of H-Ras protein (lane 3). When H-Ras was transfected with antisense to galectin-1, there was a decrease in Ras protein (lane 4), which was associated with a decrease in galectin-1 protein. The vector and the antisense controls did not result in the production of Ras protein (lanes 1 and 2).

FIG. 6 contains photographs of cells that produce H-Ras tagged with green fluorescent protein (GFP). This allows for localization of Ras in live cells using fluorescent microscopy. Top: Following transfection with GFP-H-Ras (12V)+pcDNA3, only the membrane of transformed cells was labeled with GFP tagged H-Ras as expected from previous studies [Niv et al., 1999]. Bottom: When GFP-H-Ras (12V) was transfected with galectin-1 antisense, a large fraction of the GFP-labeled Ras was displaced into the cytoplasm from the cell membrane, as the galectin-1 anchor protein was reduced.

### BEST MODE OF CARRYING OUT THE INVENTION

One aspect of the present invention is directed to a method for identifying anchor proteins for the four farnesylated isoforms of Ras, namely H-Ras, K-Ras 4A, K-Ras 4B and N-Ras, the mutated forms of which are known to be oncogenic. The Ras antagonist FTS and its active analogs are drugs containing the prenyl group farnesyl that specifically displaces activated Ras and oncogenic Ras from their binding sites on the cell membranes. These prenylated drugs are sufficient to displace and inactivate Ras even though Ras membrane binding is in part determined by the C-terminal amino acids of the Ras protein. The present invention utilizes this property to identify and isolate specific binding sites or anchor proteins for farnesylated Ras.

Proteins such as Ras and its anchors are closely associated in cell membranes and can be chemically joined together by a cross-linking agent such as disuccininimidyl subarate (DSS) and dithiobis (succinirnidyl proprionate) (DSP). The crossed linked Ras/anchor protein(s) complexes are larger and will migrate slowly on SDS gels compared to Ras. Candidate molecules for isolation are complexes that stain with anti-Ras antibodies, that are larger than Ras, and that are present in markedly reduced concentrations in membranes that are pretreated with the Ras antagonists. Once the cross-linked complex is purified, the candidate anchor protein for the farnesylated Ras is released from Ras by reversing the chemical cross-linking and isolated such as by fractionation on a SDS gel. Separation of the Ras protein from the putative anchor is conducted in accordance with standard techniques such as liquid chromatography and gel electrophoresis. The released anchor protein may then be extracted from the gel and sequenced. This approach may also be used to identify the anchors for all other isoforms of Ras.

In other embodiments, anchor proteins for isoforms of Ras that are prenylated with groups other than farnesyl as well as those that are not prenylated are identified. FTS and its active prenylated analogs also competitively displace regulatory proteins that are anchored to the cell membrane by a prenyl group other than farnesyl such as geranyl geranyl. Suitable analogs of FTS include 5-fluoro-FTS, 5-chloro-FTS, 4-chloro-FTS, 2-chloro-5-farnesylaminobenzoic acid, 3-farnesylthio-cis-acrylic acid, farnesyl thionicoatinic acid, or S-farnesyl-methylthiosalicylic acid. These prenylated drugs together with cross-linking reagents permit isolation and identification of anchor proteins for prenylated proteins such as Rac and Rho. Other Ras associated regulatory proteins such as Rit, Rin and many nonprenylated isoforms of Ras are bound to cell membranes without the aid of a prenyl group. The interaction of these latter regulatory proteins with the cell membrane is dependent upon a small portion of their amino acid sequences. Nonetheless, small organic molecules that interact with these amino acid sequences can completely displace these proteins from their membrane anchor sites. Thus, the anchor sites are identified using the combination of the displacing drug and cross-linking reagents. Suitable organic molecules can be identified from large chemical libraries.

These methods, as well as other methods disclosed herein, may be conducted using whole cells, cell lysate or homogenate, or isolated cell membranes or fragments thereof. Preferred whole cells include NIH fibroblasts transformed with oncogenic K-Ras 4B (12V), H-Ras (12V) or N-Ras (13V), 518A2/N-Ras melanoma cells, 607B melanoma cells, Panc-I cells containing oncogenic K-Ras, transformed Rat-1 EJ cells and MC-MA-11 cells.

Another aspect of the present invention is directed to methods for screening chemicals for identification of drugs that block the interactions of Ras isoforms or proteins with their cognate anchor proteins. The methods identify molecules that displace a regulatory protein such as Ras from any cellular anchorage site. The functions of such anchorage proteins are to allow the regulatory proteins such as Ras to interact with cellular membranes so that they can dimerize and combine with cytosplasmic factors to enhance or propagate their activities. Thus, the anchor protein may be obtained from any cellular compartment. In general, the methods involve the competitive inhibition of the interaction of two proteins (e.g., the Ras isoform and anchor protein) by the drug candidate. As a result, any competitive binding assay that involves interaction of three or four components may be employed. Many of these assays have been developed to measure hormones in biological fluids, hormone receptor interactions, and antibody/antigen interactions and interaction of regulatory proteins with activators and suppressors. Such binding reactions are usually made at equilibrium or in real time depending on the instrurnentation. In each instance, the endpoint of the assay directly or indirectly measures the interaction of the drug with one or both proteins or quantifies the biological consequences of this interaction. Depending upon the particular method employed, the anchor protein and/or the Ras protein is immobilized on a matrix or is in solution. In addition, either or both proteins may be detectably labeled e.g., with a fluorescent protein such as green fluorescent protein (GFP) or yellow fluorescent protein, such as when movement of the protein(s) from one location within the cell to another is being observed.

In one embodiment, the effect of the drug candidate on the interaction between the Ras protein and the anchor protein is determined by measuring the extent to which dimer formation of Ras protein is reduced. For Ras and its isoforms to be active, they must be recruited to the cell membrane where they form dimers in association with their anchors. The Ras dimer then interacts with and activates Raf protein and other cytoplasmic factor(s). This complex then initiates a regulatory cascade. The drug disruption of dimer formation or the recruitment of other molecules such as Raf is quantified. Methods for quantification of Ras dimer formation and Raf activity e.g., by determining binding of Raf to Ras, are described in Inouye, et al., (2000). In another embodiment, the effect of the drug candidate on the interaction between the Ras protein and the anchor protein is determined by measuring the extent to which cross-linking of the Ras protein with the anchor protein is reduced. Samples of Ras/anchor complex are reacted with and without the drug candidate followed by treatment with a cross linking agent. The amount of complexation with and without the drug is measured.

In yet another embodiment, the effect on the interaction is determined by measuring the extent of Ras binding with the anchor protein. The method may be conducted by observing movement of protein in a living cell. Further, the method may be conducted with both proteins in solution or wherein one of the proteins may be immobilized on a matrix such as a column. The proteins are identified in accordance with standard techniques, such as by an antibody, a fluorescent tag, or by protein-protein interaction. Examples of protein interactions include: (i) an affinity column or membrane with one protein coupled to the matrix and the drug prevents binding of the second protein; (ii) surface plasmon resonance e.g., as measured with “BIAcore” biosensor instrumentation where one protein in solution interacts with the other protein anchored to the cell of the Biocor, wherein protein/protein interactions and the ability of drugs to disrupt such interactions are measured in real time (an advantage of this technology being that affinity constants can be measured); (iii) color transfer generated by interaction of two tagged proteins in solution and how this is influenced by drugs is also measured in real time; (iv) interaction of a fluorescent tagged protein with an untagged protein which is coated on the surface of an object such as a sheep red blood cells calows measurement of drug induced interactions with a FACScan machine (Guava Personal Cytometer); and (v) interaction of a tagged protein in a microtiter plate which allows drug modulation of protein/protein interaction at equilibrium using a microtiter plate reader.

In preferred embodiments, the method is conducted using galectin-1, galectin-3, galectin-7 or galectin-8. Galectin-1 is a protein known to bind beta-galactoside. The amino acid sequence of the rat protein is as follows: MACGLVASNLNLKPGECLKVRGELAPDAKSFVLNLGKDSNNLCLHFNPRFNA HGDANTIVCNSKDDGTWGTEQRETAFPFQPGSITEVCITFDQADLTIKLPDGHE FKFPNRLNMEAINYMAADGDFKIKCVAFE (SEQ ID NO:1). See, Clerch, et al. (1988). The corresponding nucleotide sequence is

(SEQ ID NO:2)

5′ATGGCCTGTGGTCTGGTCGCCAGCAACCTGAATCTCAAACCTGGGGAA
TGTCTCAAAGTTCGGGGAGAGCTGGCCCCGGACGCCAAGAGCTTTGTGTT
GAACCTGGGGAAAGACAGCAACAACCTGTGCCTACACTTCAACCCCCGCT
TCAACGCCCACGGAGATGCCAACACCATTGTGTGTAACAGCAAGGACGAT
GGGACCTGGGGAACAGAACAACGGGAGACTGCCTTCCCTTTCCAGCCTGG
GAGCATCACGGAGGTGTGCATCACCTTTGACCAGGCTGACCTGACCATCA
AGCTGCCAGACGGGCATGAATTCAAATTCCCCAACCGCCTCAACATGGAG
GCCATCAACTACATGGCGGCGGATGGTGACTTCAAGATTAAGTGTGTGGC
CTTTGAGTGA 3′

Galectin-1 obtained from mouse and human cells has also been reported. See, Wilson, et al. (1989) and Gitt, et al. (1991). The amino acid and corresponding nucleotide sequences of human galectin-1 are set forth as SEQ ID NOS: 3 and 4 respectively.

(SEQ ID NO:3)

MACGLVASNLNLKPGECLRVRGEVAPDAKSFVLNLGKDSNNLCLHFNPRF
NAHGDANTIVCNSKDGGAWGTEQREAVFPFQPGSVAEVCITFDQALNLTV
KLP DGYEFKFPNRINLEATNYMAADGDFKIKCVAFD

Coding sequence:

(SEQ ID NO:4)

5′ ATGGCTTGTGGTCTGGTCGCCAGCAACCTGAATCTCAAACCTGGAGA
GTGCCTTCGAGTGCGAGGCGAGGTGGCTCCTGACGCTAAGAGCTTCGTGC
TGAACCTGGGCAAAGACAGCAACAACCTGTGCCTGCACTTCAACCCTCGC
TTCAACGCCCACGGCGACGCCAACACCATCGTGTGCAACAGCAAGGACGG
CGGGGCCTGGGGGACCGAGCAGCGGGAGGCTGTCTTTCCCTTCCAGCCTG
GAAGTGTTGCAGAGGTGTGCATCACCTTCGACCAGGCCAACCTGACCGTC
AAGCTGCCAGATGGATACGAATTCAAGTTCCCCAACCGCCTCAACCTGGA
GGCCATCAACTACATGGCAGCTGACGGTGACTTCAAGATCAAATGTGTGG
CCTTTGACTGA 3′

Galectin-1 DNAs from the mouse and rat are not identical but they possess 94% sequence similarity. The corresponding amino acid sequences possess 95% sequence similarity. The amino acid sequence of the mouse galectin-1 is as follows:

(SEQ ID NO:5)

MACGLVASNLNLKPGECLKVRGEVASDAKSFVLNLGKDSNNLCLHFNPPY
NAHGDANTIVCNTKEDGTWGTEHREPAFPFQPGSITEVCITFDQADLTIK
LPDGHEFKFPNRLNMEATNYMAADGDFKIKCVAFE

The molecular weight of galectin-3 varies within and among species and ranges from 29-34 kD. See, Liu, et al. (1987) (rat); Pillai, (1990) (human); and Cherayil, et al. (1989) (mouse). The amino acid and corresponding nucleic acid sequences for two human galectin-3 proteins are set forth below.

| | |
|---|---|
| MADNFSLHDALSGSGNPNPQGWPGAWGNQPAGAGGYPGA SYPGAYPGQAPPGAYPGQAPP | (SEQ ID NO:6) |
| GAYPGAPGAYPGAPAP GVYPGPPSGPGAYPSS GQPSATGAYPATGPYGAPAGPLTVPYNLPL | |
| PGG VVPRMLITILGTVKPNA NRIALDFQRGNDVAFHFN PRFNENNRRVIVCNTKLDNNWGR | |
| BERQ SVFPFESGK PFKIQVLVEPDHFKVAVNDAHLLQYNHRVKK LNEISKLGISGDIDLTS | |
| ASYTMI | |
| Coding sequence: | |
| 5′ ATGGCAGACAATTTTTC GCTCCATGAT GCGTTATCTG GGTCTGGAAA CCCAAACCCT | (SEQ ID NO:7) |
| CAAGGATGGCCTGGCGCATG GGGGAACCAG CCTGCTGGGG CAGGGGGCTA CCCAGGGGCT | |
| TCCTATCCTGGGGCCTACCC CGGGCAGGCA CCCCCAGGGG CTTATCCTGG ACAGGCACCT | |
| CCAGGCGCCTACCCTGGAGC ACCTGGAGCT TATCCCGGAG CACCTGCACC TGGAGTCTAC | |
| CCAGGGCCACCCAGCGGCCC TGGGGCCTAC CCATCTTCTG GACAGCCAAG TGCCACGGGA | |
| GCCTACCCTGCCACTGGCCC CTATGGCGCC CCTGCTGGGC CACTGATTGT GCCTTATAAC | |
| CTGCCTTTGCCTGGGGGAGT GGTGCCTCGC ATGCTGATAA CAATTCTGGG CACGGTGAAG | |
| CCCAATGCAAACAGAATTGC TTTAGATTTC CAAAGAGGGA ATGATGTTGC CTTCCACTTT | |
| AACCCACGCTTCAATGAGAA CAACAGGAGA GTCATTGTTT GCAATACAAA GCTGGATAAT | |
| AACTGGGGAAGGGAAGAAAG ACAGTCGGTT TTCCCATTTG AAAGTGGGAA ACCATTCAAA | |
| ATACAAGTACTGGTTGAACC TGACCACTTC AAGGTTGCAG TGAATGATGC TCACTTGTTG | |
| CAGTACAATCATCGGGTTAA AAAACTCAAT GAAATCAGCA AACTGGGAAT TTCTGGTGAC | |
| ATAGACCTCACCAGTGCTTC ATATACCATG ATATAA 3′ | |
| B001120. Homo sapiens, lec . . . [gi:12654570] | |
| MADNFSLHDALSGSGNPNPQGWPGAWGNQPAGAGGYPGASYPGAYPGQAPPGAYPGQAPPGAYP | (SEQ ID NO:8) |
| GAPGAYPGAPGVYPGPPSGPGAYPSSGQPSATGAYPATGPYGAPAGPLIVPYNLPLPG GVVPR | |
| MLITILGTV KPNAINRLALDFQRGNDVAFKFNPRFNENNRRVIVCNTKLDNNWGREERQSVFP | |
| FESGKPFKIQVLVEPDHFKVAVNDAHLLQYNHRVKKLNEISKLGISGDIDLTSASYTMI | |
| Coding sequence: | |
| 5′ ATGGCAG ACAATTTTTC GCTCCATGATGCGTTATCTG GGTCTGGAAA CCCAAACCCT | (SEQ ID NO:9) |
| CAAGGATGGC CTGGCGCATG GGGGAACCAGCCTGCTGGGG DAGGGGGCTA CCCAGGGGCT | |
| TCCTATCCTG GGGCCTACCC CGGGCAGGCACCCCCAGGGG CTTATCCTGG ACAGGCACCT | |
| CCAGGCGCCT ACCCTGGAGC ACCTGGAGCTTATCCCGGAG CACCTGCACC TGGAGTCTAC | |
| CCAGGGCCAC CCAGCGGCCC TGGGGCCTACCCATCTTCTG GACAGCCAAG TGCCACCGGA | |
| GCCTACCCTG CCACTGGCCC CTATGGCGCCCCTGCTGGGC CACTGATTGT GCCTTATAAC | |
| CTGCCTTTGC CTGGGGGAGT GGTGCCTCGCATGCTGATAA CAATTCTGGG CACGGTGAAG | |
| CCCAATGCAA ACAGAATTGC TTTAGATTTCCAAAGAGGGA ATGATGTTGC CTTCCACTTT | |
| AACCCACGCT TCAATGAGAA CAACAGGAGAGTCATTGTTT GCAATACAAA GCTGGATAAT | |
| AACTGGGGAA GGGAAGAAAG ACAGTCGGTTTTCCCATTTG AAAGTGGGAA ACCATTCAAA | |
| ATACAAGTAC TGGTTGAACC TGACCACTTCAAGGTTGCAG TGAATGATGC TCACTTGTTG | |
| CAGTACAATC ATCGGGTTAA AAAACTCAATGAAATCAGCA AACTGGGAAT TTCTGGTGAC | |
| ATAGACCTCA CCAGTGCTTC ATATACCATGATATAA 3′ | |

Three additional human galectin-3 sequences, as well as a rat and mouse galectin-3 sequence, are set forth below.

| | |
|---|---|
| M35368 (human) | |
| MADNFSLHDALSGSGNPNPQGWPGAWGNQPAGAGGYPGASYPGA | (SEQ ID NO:10) |
| YPGQAPPGAYPGQAPPGAYPGALPGAYPGAYAPGVYPGPPSGPGAYPSSGQPSA | |
| PGAYPATGPYGAPAGPLIVPYNLPLPGGVVPRMLITILGTVKPN | |
| ANRIALDFQRGNDVAFHFN PRFNENNRRVIVCNTKIDNNW | |
| GREERQSVFPFESGKPFKIQVLVEPDHFKVAVNDAIHL | |
| LQYNIIRVKKLNEISKLGISGDIDLTSASYTMI | |
| NM_002306 (human) | |
| MADNFSLHDALSGSGNPNPQGWPGAWGNQPAGAGGYPGASYPGA | (SEQ ID NO:11) |
| YPGQAPPGAYPGQAPPGAYHGAPGAYPGAPAPGVYPGPPSGPGAYPSSGQPSAPGAYP | |
| ATGPYGAPAGPLTVPYNLPLPGGVVPRMLITILGTVKPNANRIALDFQRGNDVAFHFN | |
| PRFNENNRRVIVCNTKLDNNWGREERQSVFPFESGKYFKIQVLVEPDHFKVAVNDAHL | |
| LQYNHRVKIKLNEISKLGISGDIDLTSASYTMI | |
| S59012 (human) | |
| MADNFSLHDALSGSGNPNPQGWPGAWGNQPAGAGGYPGASYPGA | (SEQ ID NO:12) |
| YPGQAPPGAYPGQAPPGAYPGAPGAYPGAPAPGVYPGPPSGPGAYPSSGQPSATGAYP | |
| ATGPYGAPAGPLIVPYNLPLPGGVVPRMLITILGTVKPNANRIALDFQRGNDVAFHFN | |
| PRFNENNRRVIVCNTKLDNNWGREERQSVFPFESGKPFKIQVLVEPDPYKVAVNDAHL | |
| LQYNHRVKKLNEISKLGISGDIDLTSASYTMI | |
| P08699 (rat) | |
| MADGFSLNDA LAGSGNPNPQ GWPGAWGNQP GAGGYPGASY PGAYPGQAPP | (SEQ ID NO:13) |
| GGYPGQAPPS AYPGPTGPSA YPGPTAPGAY PGPTAPGAFP GQPGGPGAYP | |
| SAPGAYPSAP GAYPATGPFG APTGPLTVPY DMPLPGGVMP RMLITIIGTV | |
| KPNANSITLN FKKGNDIAFH FNPRFNENNR RVIVCNTKQD NNWGREERQS | |
| AFPFESGKPF KIQVLVEADH FKVAVNDVHL LQYNHRMKNL REISQLGIIG | |
| DITLTSASHA MI | |
| P16110 (mouse) | |
| MADSFSLNDA LAGSGNPNPQ GYPGAWGNQP GAGGYPGAAY PGAYPGQAPP | (SEQ ID NO:14) |
| GAYPGQAPPG AYPGQAPPSA YPGPTAPGAY PGPTAPGAYP GQPAPGAFPG | |
| QPGAPGAYPQ CSGGYPAAGP GVPAGPLTV PYDLPLPGGV MPRMLITIMG | |
| TVKPNANRIV LDFRRGNDVA FHFNPRFNEN NRRVIVCNTK QDNNWGKEER | |
| QSAFPFESGK PFKIQVLVEA DHFKVAVNDA HLLQYNHRMK NLREISQLGI | |
| SGDITLTSAN HAMI | |

In other embodiments, the method is conducted using galectin-7 and/or galectin-8, which Applicants have also found to function as cell membrane anchors for Ras isoforms. Amino acid and corresponding nucleic acid sequences of human galectin-7 and -8 are set forth below.

Galectin-7

| | |
|---|---|
| L07769. Homo sapiens galectin-7 [gi:182131] | |
| MSNVPHKSSLPEGTRPGTVLRIRGLVPPNASRFHVNLLCGEEQG | (SEQ ID NO:15) |
| SDAALHFNPRLDTSEVVFNSKEQGSWGREERGPGVPFQRGQPFEVLIIASDDGF | |
| KAVVGDAQYHHFRHRLPLARVRLVEVGGDVQLDSVRIF | |
| Coding sequence: | |
| 5′ATGTCCAACGTC CCCCACAAGT CCTCGCTGCC CGAGGGCATCCGCCCTGGCA CGGTGCTGAG | (SEQ ID NO:16) |
| AATTCGCGGC TTGGTTCCTC CCAATGCCAG CAGGTTCCATGTAAACCTGC TGTGCGGGGA | |
| GGAGCAGGGC TCCGATGCCG CCCTGCATTT CAACCCCCGGCTGGACACGT CGGAGGTGGT | |
| CTTCAACAGC AAGGAGCAAG GCTCCTGGGG CCGCGAGGAGCGCGGGCCGG GCGTTCCTTT | |
| CCAGCGCGGG CAGCCCTTCG AGGTGCTCAT CATCGCGTCAGACGACGGCT TCAAGGCCGT | |
| GGTTGGGGAC GCCCAGTACC ACCACTTCCG CCACCGCCTGCCGCTGGCGC GCGTGCGCCT | |
| GGTGGAGGTG GGCGGGGACG TGCAGCTGGA CTCCGTGAGGATCTTCTGA 3′ | |
| Galectin-8 | |
| AY037304. Homo sapiens beta . . . [gi:14626473] | |
| MMLSLNNLQNIIYSPVIPYVGTIPDQLDPGTLTVICGHVPSDAD | (SEQ ID NO:17) |
| RFQVDLQNGSSVKPRDVAYHFNPRFKPAGCTVCNTLTNEKWGBEEITYDTPFK | |
| REKSFEIVIMVLKDKFQ VPKSGTPQLPSNRGGDISKIAPRTVYTKSKD S TV | |
| NHTLTCTKIPPTNYVSKILPFAALNTPMGPGGTVVVKGEVNANAK | |
| SFNVDLLAGKSKHIALHLNPRLNIKAFVRNSFLQESWGEEEPNITS | |
| FPFSPGMYFEMIIYCDVREFKVAVNGVHSLEYKHR | |
| FKELSSIDTLEINGDIHLLEVRSW | |
| Coding sequence: | |
| 5′ATGATGTTGT CCTTAAACAA CCTACAGAAT ATCATCTATA GCCCGGTAAT CCCGTATGTT | |
| GGCACCATTC CCGATCAGCT GGATCCTGGA ACTTTGATTG TGATATGTGG GCATGTTCCT | |
| AGTGACGCAG ACAGATTCCA GGTGGATCTG CAGAATGGCA GCAGTGTGAA ACCTCGAGCC | |
| GATGTGGCCT TTCATTTCAA TCCTCGTTTC AAAAGGGCCG GCTGCATTGT TTGCAATACT | |
| TTGATAAATG AAAAATGGGG ACGGGAAGAG ATCACCTATG ACACGCCTTT CAAAAGAGAA | |
| AAGTCTTTTG AGATCGTGAT TATGGTGCTA AAGGACAAAT TCCAGGTTCC AAAGTCTGGC | |
| ACGCCCCAGC TTCCTAGTAA TAGAGGAGGA GACATTTCTA AAATCGCACC CAGAACTGTC | |
| TACACCAAGA GCAAAGATTC GACTGTCAAT CACACTTTGA CTTGCACCAA AATACCACCT | |
| ACGAACTATG TGTCGAAGAT CCTGCCATTC GCTGCAAGGT TGAACACCCC CATGGGCCCT | |
| GGCGGCACTG TCGTCGTTAA AGGAGAAGTG AATGCAAATG CCAAAAGCTT TAATGTTGAC | |
| CTACTAGCAG GAAAATCAAA GCATATTGCT CTACACTTGA ACCCACGCCT GAATATTAAA | |
| GCATTTGTAA GAAATTCTTT TCTTCAGGAG TCCTGGGGAG AAGAAGAGAG AAATATTACC | |
| TCTTTCCCAT TTAGTCCTGG GATGTACTTT GAGATGATAA TTTATTGTGA TGTTAGAGAA | |
| TTCAAGGTTG CAGTAAATGG CGTACACAGC CTGGAGTACA AACACAGATT TAAAGAGCTC | |
| AGCAGTATTG ACACGCTGGA AATTAATGGA GACATCCACT TACTGGAAGT AAGGAGCTGG | |

TAG 3′ (SEQ ID NO:18). In the methods of the present invention, fragments of the anchor proteins that bind the Ras protein may also be used. Thus, the term “anchor protein” as used herein includes such fragments as well as the full-length proteins.

Another aspect of the present invention is directed to a method for reducing or inhibiting aberrant Ras activity in vivo. In general, aberrant Ras activity is manifested by uncontrolled mitosis. Diseases characterized by this phenomenon include cancers and various non-malignancies such as autoimmune diseases (e.g., type 1 diabetes, lupus and multiple sclerosis), cirrhosis, graft rejection, atherosclerosis, polycystic kidneys and post-angioplasty restenosis. Preferred indications are diseases characterized by proliferation of the cells of the diseased organ, including a proliferation of T-cells. The method entails administering to patients oligonucleotides that are in the antisense orientation to the mRNAs for galectin-1, galectin-3 and/or another Ras anchor protein such as galectin-7 or galectin-8. They are designed based on sequences that show the most potent effects on translation of the protein and minimizing non-antisense effects.

Factors taken into consideration in the design of antisense DNAs include the length of an oligonucleotide, its binding affinity and accessibility of the target RNA, resistance to degradation by endogenous nucleases, permeability through target cell membranes. Tens of oligonucleotides may be screened on target cells in culture to select the most potent inhibitors (Wagner, et al., 1993). Tumor cells are particularly preferred target cells. In general, most regions of the RNA (e.g., 5′- and 3′-untranslated, AUG initiation sites, splice junctions and introns) may be targeted using antisense oligonucleotides. Enhanced binding affinity and nuclease stability are critical for antisense activity. Optimal length of the oligonucleotides varies, but in general, is about 15 nucleotides. The sequence of an antisense compound does not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed. The inclusion of phosphorathioate-modified oligonucleotides that contain the C-5 propyne analogs of uridine and cytidine improve binding and stability of the antisense oligos. (Wagner, 1993). Modest increases in activity can also be achieved by delivering the antisense oligos via a liposome. For example, up to a 10-fold increase in biological activity of oligonucleotides in vitro is achieved by complexing with the serum resistant cationic liposome, GS2888. See also WO 96/40062 which discloses methods for encapsulating high molecular weight nucleic acids in liposomes; U.S. Pat. No. 5,264,221 which discloses protein-bonded liposomes and asserts that the contents of such liposomes may include an antisense RNA; U.S. Pat. No. 5,665,710 which describes certain methods of encapsulating oligodeoxynucleotides in liposomes; WO 97/04787 which discloses liposomes comprising antisense oligonucleotides targeted to the raf gene.

In preferred embodiments, the oligonucleotides are formulated for human use by dissolution in a saline solution for IV administration. A dose response effect is expected at doses between 0.06 and 7 mg/kg/day for one to two weeks of continuous treatment (Wagner, 1995). The antisense oligonucleotides bind the nucleic acid e.g., mRNA, of the anchor protein, thus causing degradation of the mRNA and secondarily causing a decrease in the concentration of Ras. The antisense compounds containing the oligonucleotides are prepared in accordance with known procedures such as those referenced in U.S. Pat. No. 6,294,382.

Antisense mRNA that bind galectin-1 mRNA, for example, may be designed by testing sequences selected along the length of the antisense mRNA and testing them in vitro for potency before using them in vivo. The full-length antisense for human galectin-1 is set forth below.

TCAGTCAAAGGCCACACATTTGATCTTGAAGTCACCGTCAGCTGCCATGTA GTTGATGGCCTCCAGGTTGAGGCGGTTGGGGAACTTGAATTCGTATCCATC TGGCAGCTTGACGGTCAGGTTGGCCTGGTCGAAGGTGATGCACACCTCTGC AACACTTCCAGGCTGGAAGGGAAAGACAGCCTCCCGCTGCTCGGTCCCCCA GGCCCCGCCGTCCTTGCTGTTGCACACGATGGTGTTGGCGTCGCCGTGGGC GTTGAAGCGAGGGTTGAAGTGCAGGCACAGGTTGTTGCTGTCTTTGCCCAG GTTCAGCACGAAGCTCTTAGCGTCAGGAGCCACCTCGCCTCGCACTCGAAG GCACTCTCCAGGTTTGAGATTCAGGTTGCTGGCGACCAGACCACAAGCCAT (SEQ ID NO:19). Preferred galectin-1 antisense oligonucleotides are as follows:

AAGTCACCGTCAGCTGCCATGTAGT (SEQ ID NO:20);

GATGCACACCTCTGCAACACTTC (SEQ ID NO:21);

TCAGCACGAAGCTCTTAGCGTCAG (SEQ ID NO:22);

GCACTCGAAGGCACTCTCCAGG (SEQ ID NO:23); and

GGTTGCTGGCGACCAGACCACA (SEQ ID NO:24).

The full-length antisense for human galectin-3 is set forth below. TTATATCATGGTATATGAAGCACTGGTGAGGTCTATGTCACCAGAAATTCC CAGTTTGCTGATTTCATTGAGTTTTTTAACCCGATGATTGTACTGCAACAGT GAGCATCATTCACTGCAACCTTGAAGTGGTCAGGTTCAACCAGTACTTGTA TTTTGAATGGTTTCCCACTTTCAAATGGGAAAACCGACTGTCTTTCTTC CCT TCCCCAGTTATTATCCAGCTTTGTATTGCAAACAATGACTCTCCTGTTGTTCT CATTGAAGCGTGGGTTAAAGTGGAAGGCAACATCATTCCCTCTTTGGAAAT CTAAAGCAATTCTGTTTGCATTGGGCTTCACCGTGCCCAGAATTGTTATCAG CATGCGAGGCACCACTCCCCCAGGCAAAGGCAGGTTATAAGGCACAATCA GTGGCCCAGCAGGGGCGCCATAGGGGCCAGTGGCAGGGTAGGCTCCGGGG GCACTTGGCTGTCCAGAAGATGGGTAGGCCCCAGGGCCGCTGGGTGGCCCT GGGTAGACTCCAGGTGCGGTGCTCCGGGATAAGCTCCAGGTGCTCCATGGT AGGCGCCTGGAGGTGCCTGTCCAGGATAAGCCCCTGGGGGTGCCTGCCCGG GGTAGGCCCCAGGATAGGAAGCCCCTGGGTAGCCCCCTGCCCCAGCAGGCT GGTTCCCCCATGCGCCAGGCCATCCTTGAGGGTTTGGGTTTCCAGACCCAG ATAACGCATCATGGAGCGAAAAATTGTCTGCCAT (SEQ ID NO:25). Preferred galectin-3 antisense oligonucleotides are as follows:

TATATGAAGCACTGGTGAGGTC (SEQ ID NO:26);

GAAGCGTGGGTTAAAGTGGAAGGC (SEQ ID NO:27);

TTGTTATCAGCATGCGAGGCACCACTCCCC (SEQ ID NO:28);

CACTTGGCTGTCCAGAAGATG (SEQ ID NO:29);

GATAAGCTCCAGGTGCTCCATGGTAG (SEQ ID NO:30); and

TCCAGACCCAGATAACGCAT (SEQ ID NO:31).

Preferred antisense oligonucleotides that bind galectin-7 and galectin-8 mRNA are as follows: Galectin-7 antisense oligo:

5′ TGTGGGGGACGTTGGACAT 3′ (SEQ ID NO:32)

Galectin-8 antisense oligo:

5′ TGTTTAAGGACAACATCAT 3′ (SEQ ID NO:33).

Yet another aspect of the present invention is directed to a method of determining efficacious dosages of a Ras antagonist that disrupts Ras-anchor protein binding. In general, the method entails contacting cells with the antagonist in vivo or in vitro, collecting the cells following the contacting, isolating cell membranes from the collected cells, measuring decrease in anchor protein concentration per unit of cell membrane protein, and correlating the decrease with dosage of the Ras antagonist. In a preferred embodiment, a method for measuring the biological action of FTS and its analogs in vivo and in vitro is based on the suppression of the immunoassayable galectin-1 in H-Ras-transformed tumors. The basis of this assay depends upon the dose dependent loss of galectin-1 from cell membranes by FTS. Under maximal stimulation of tumor cells with FTS, 90% of galectin-1 is lost from membrane. This allows for an excellent dose response. A variation of this assay uses drug induced suppression of the anchor proteins in normal human lymphocytes isolated from patients being treated with FTS in phase 1 clinical trials. A dose of FTS or any other Ras antagonist that maximally suppresses galectin-1 (or the respective anchor protein of the antagonist) should be a dose that produces effects on other biological endpoints in vivo. When such assay is in mice and humans, therapeutically efficacious doses of Ras antagonists for humans can be determined.

Various aspects of the present invention are further illustrated by the following examples. The presentation of these examples is by no way intended to limit Applicants' invention in any way. Unless otherwise specified, all percentages are by weight.

### EXAMPLE 1

We used chemical cross-linkers to isolate a protein whose interaction with Ras could be blocked by FTS. We identified such a protein that forms FTS-sensitive complexes with H-Ras (12V) in transformed Rat-1 (EJ) cells. This protein was isolated from such complexes and identified by mass spectrophotometry (MS) and by specific antibodies such as galectin-1, a mammalian galactose-binding protein known to be associated with cell growth and transformation. Cross-liking of H-Ras to galectin-1 detected in intact EJ cells and in cell membranes was independent of galectin-1 sugar-binding activity. FTS (but not its inactive analog, GTS) decreased the levels of endogenous galectin-1 in EJ cells in parallel with the decrease in Ras. Galectin-1 seems to interact preferentially with farnesylated H-Ras (12V). K-Ras 4B (12V) did interact with galectin-1 though less efficiently than H-Ras. Galectin-3 interacted with K- and H-Ras. Activated N-Ras (13V) did not form complexes with galectin-1 or galectin-3. Co-expression of galectin-1 antisense RNA and H-Ras (12V) in two cell lines resulted in a decrease in Ras protein as detected by Western blots. Using confocal microscopy expression of galectin-1 antisense resulted in release of H-Ras labeled with green fluorescent protein (GFP) from the membranes of live cells. Thus, H-Ras (12V) and galectin-1 seem to interact in the cell membrane and to cooperate in cell transformation. These results provide a link between Ras transformation and the known sugar-independent mitogenic and transforming potentials of galectin-1, which, like those of activated Ras, are associated with many types of human malignancies.

1. Identification of Ras-Interacting Proteins Sensitive to the Ras Inhibitor FTS

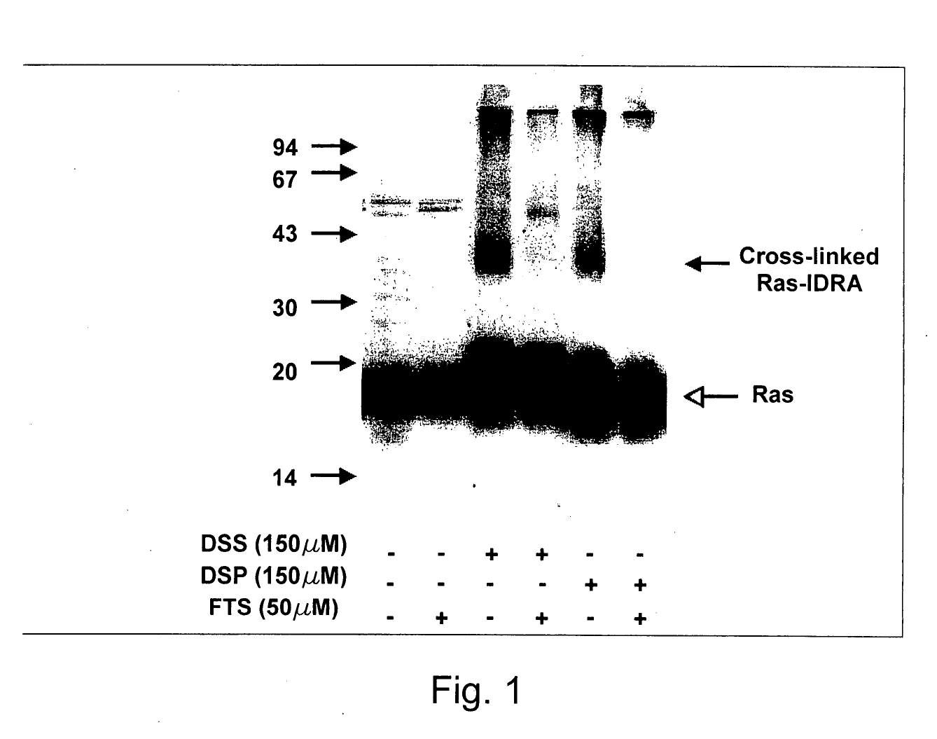

The somewhat limited, but fast, lateral mobility of Ras in the cell membrane suggests that interactions of Ras with other proteins are likely to be dynamic and transient [Niv et al., 1999]. We used chemical cross-linkers in an attempt to identify the rapidly dissociating complexes of Ras and Ras-interacting proteins. The Ras inhibitor FTS, which was shown to relieve constraints on the lateral mobility of Ras [Niv et al., 1999], was used as an analytical tool in order to identify complexes that are sensitive to this inhibitor. Accordingly, the analytical steps were performed with control and with FTS-treated EJ cells in combination with the cross-linkers DSS and DSP, the latter of which is reducible. When membranes of control and FTS-treated cells were exposed to these cross-linkers solubilized and fractionated on SDS-containing gels, Ras-immunoreactive bands were clearly detected at 34-43 kDa, 50 kDa, and 70 kDa (FIG. 1). These complexes were not detected in the absence of the cross-linkers. The broadband at 34-43 kDa was not present in cells (data not shown) or cell membranes (See FIG. 1) after treatment with FTS. The proteins in this band fit the criterion of Ras proteins bound to their specific anchors (IDRA) because FTS and its active analogs prevent this binding. Interaction of Ras with the IDRAs was not disrupted with analogs of FTS that had no anti-Ras activity on tumor cells (data not shown). This experiment showed how to identify proteins that interact with Ras and an anti-Ras drug.

2. Purification of Ras-Interacting (Anchor) Proteins from EJ Cells.

Triton X-100 extracts of the membranes containing Ras complexes formed by cross-linking with DSP were used for subsequent purification steps. The first steps were performed in the absence of reducing reagents to enable the purification to be followed by Ras antibodies. The release of Ras from the putative IDRA proteins was performed only at the final fractionation step. The details of the purification are summarized by way of the following flow diagram.

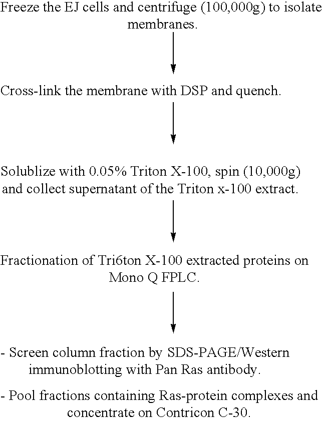

Purify 34-43 kDa Ras-protein complexes.

Run concentrate Mono Q pool on SDS-PAGE and stain with coomassie blue. Cut 34-43 kDa-wide band.

Extract bands with SDS sample buffer and divide each sample into two portions.

Run samples on second SDS gel: one portion of each sample without DTT and the other with DTT.

Identify putative IDRA proteins with silver staining.

FPLC MonoQ ion exchange chromatography yielded an enriched preparation of Ras-protein complexes. The above noted 34-43 kDa band appeared to be the most prominent one. Complexes with higher molecular weights were enriched as well. Ras and all species of the Ras-immunoreactive complexes detected in the pooled MonoQ fractions could be specifically immunoprecipitated by biotin-pan Ras antibody. Assuming that the larger complexes may represent multiples of the 34-43 kDa complexes, the Ras-immunoreactive band with the lowest molecular weight was further purified. Two consecutive gel purification steps were used. Following the first gel prepared under non-reducing conditions, a gel slice corresponding to 34-43 kDa proteins was excised from the gel and the proteins were then extracted with SDS sample buffer in the absence or in the presence of a reducing reagent (DTT). As expected, under non-reducing conditions only the 34-43 kDa Ras-immunoreactive band was detected by Western immunoblotting with Ras antibody and 21 kDa Ras was released from the complexes by reduction with DTT. In addition, two major proteins were released by DTT from the 34-43 kDa Ras-immunoreactive complexes. One was a 14-15 kDa protein and the other a 19-20 kDa protein. As the sum of the apparent molecular weights of the 21 kDa H-Ras (12V) protein and each of these proteins corresponded well to the 3443 kDa complexes, both proteins seemed like reasonable candidates for Ras-interacting anchor. To further demonstrate that these proteins were good candidates for molecules that specifically interact with H-Ras (12V), the above described purification procedures were repeated, using in parallel equal numbers of EJ cells, their parental Rat-1 cells, and myr H-Ras (12V)-transformed Rat-1 cells. The amounts of both the 14-15 kDa and of the 19-20 kDa proteins were significantly lower in Rat-1 cells compared to EJ cells. The 14-15 kDa protein was barely detected in the myr H-Ras (12V) cells. These results suggested that the 14-15 kDa protein interacts with the farnesylated H-Ras and may be involved in cell transformation induced by this Ras isoform. Further experiments focused on this 14-15 kDa protein.

3. The 14-15 kDa Band was Identified as Rat Galectin-1

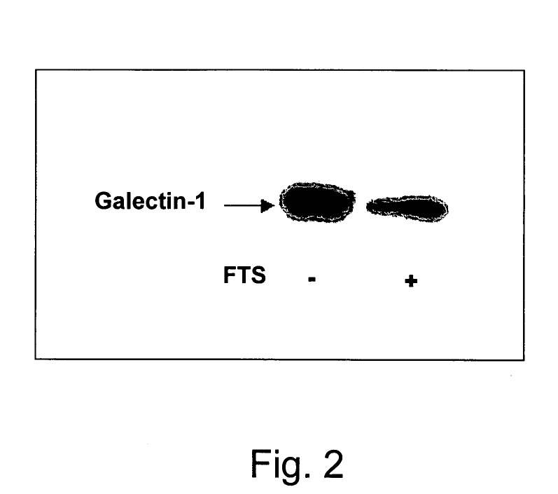

Quantitative and high degree of purification of the 14-15 kDa required several gel purification steps as described in the flow diagram. The highly purified protein released by reduction was subjected to trypsin cleavage followed by microbore HPLC separation of the tryptic fragments and MS analysis of the isolated peptides. Fragmentation patterns of two peptides corresponded precisely to the 14 kDa rat galectin-1. The fact that galectin is a 14 kDa protein (the size of the isolated protein) further confirmed that the FTS-sensitive Ras-interacting protein is galectin-1, a previously identified sugar- binding protein [Perillo et al., 1998]. Antibodies raised against an N-terminal peptide of galectin-1 confirmed this conclusion. Consistent with the early observations that the Ras inhibitor FTS inhibited the formation of the cross-linked 34-43 kDa Ras-immunoreactive band (FIG. 1), the amount of galectin-1 purified from FTS-treated EJ cells using the procedure shown in the flow diagram was very low (FIG. 2). In addition, immunoprecipitation of the 34-43 kDa Ras protein complexes with biotin-Ras antibody followed by immunoblotting with galectin-1 antibody revealed that galectin-1 is indeed part of the complex and that it is released by DTT.

To be certain that the above results were not a function of use of the cross-linking reagent on isolated membranes, control and FTS-treated cells were exposed to the cross-linker DSP. Membranes were isolated and complexes were purified by the two-step gel purification procedure described above. Slices of the first non-reducing gel, corresponding to 3443 kDa, 43-67 kDa, and 67-95 kDa proteins, were excised from the gel and subjected to the second gel in the presence of DTT. Each sample was then immunoblotted with both Ras and galectin-1 antibody. The results show that both proteins were released from complexes of all sizes. These results show that H-Ras (12V) and galectin-1 do interact in the intact cell and that they may either form complexes with additional proteins and/or form multimeric complexes. In this respect, both Ras [Inouye et al., 2000] and galectin-1 form homodimers [Perillo et al., 1998].

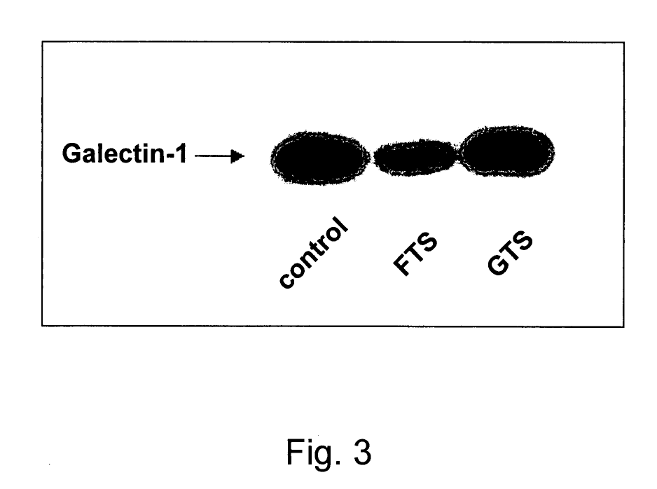

In another set of experiments, intact EJ cells were treated either with FTS or with its inactive analog GTS (without cross-linking) and the effects of the compounds on the amounts of membrane Ras and membrane galectin-1 were determined. In agreement with previous results [Kloog et al., 1999] FTS, but not GTS, reduced the amount of membrane Ras in the cells by 50-60% (data not shown). Similarly, FTS (but not GTS) induced a 90% reduction in the amount of membrane galectin-1 (FIG. 3). Thus, the magnitude of the FTS-induced galectin-1 decrease was much greater than that of Ras. This result demonstrates the utility of galectin-1 in a bioassay for FTS and its active analogs in cell culture and intact animals, including humans.

4. Galectin-1 Exhibits a Significant Specificity Towards H-Ras (12V)

Experiments were conducted to determine whether all the Ras isoforms interact with galectin-1. A comparative analysis was performed on the amounts of galectin-1 in three cell types: H-Ras (12V)-transformed Rat-1 (EJ) cells, N-Ras (13V)-transformed Rat-1 cells, and K-Ras 4B (12V)-transformed NIH 3T3 cells. In all of these cell lines, FTS is known to dislodge Ras from cell membranes [Kloog et al., 1999]. The results showed that all of these cells expressed galectin-1, yet EJ and the K-Ras 4B cells expressed higher amounts of galectin-1 compared to the N-Ras (13V) cells. Cross-linking experiments were performed with each of the cell lines to determine whether Ras and galectin-1 were released from complexes of 34-43 kDa proteins. Comparable amounts of Ras were released from complexes of all cell lines. By contrast, the amounts of galectin-1 released from the complexes were varied. Galectin-1 was very high in complexes from H-Ras-transformed EJ cells, significantly lower in the K-Ras 4B (12V) cells, and was barely detected in the N-Ras (13V) cells. Since all of the cell lines tested express galectin-1 and all over-express the corresponding Ras isoform, these results suggest that galectin-1 exhibits significant specificity toward H-Ras (12V).

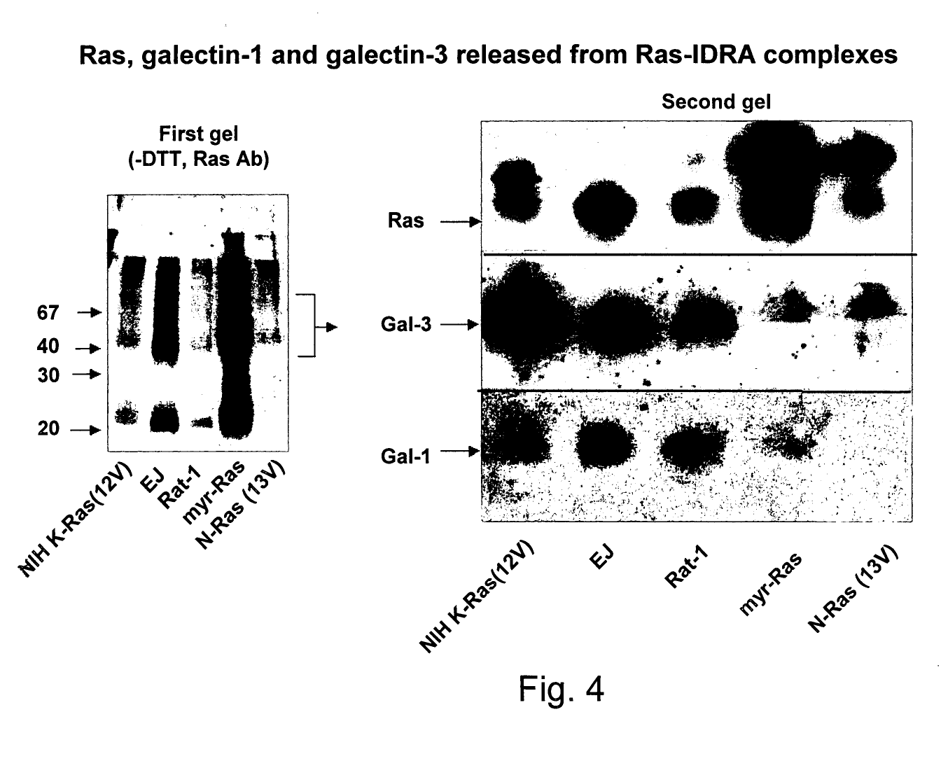

The above observations suggested that some of the isoforms of Ras may prefer other IDRAs. To examine this possibility, the above cross-linking experiment with the cell lines containing the three Ras isoforms was repeated and the release of galectin-1 and galectin-3 from the 34-43 kDa band isolated from cross-linked membranes was determined. The results shown in FIG. 4 indicate that Ras is released from all membranes. K-Ras (12V) is associated with galectin-3 and H-Ras (12V) is associated with galectin-1 and -3 in these complexes. By contrast, N-Ras (13V) anchorage to the cell membrane does not appear to be explained by either galectin-1 or -3. As expected, myristoylated Ras is not associated with an anchor protein as it is attached to the membrane by a different mechanism. The Ras in untransformed cells (Rat 1) uses galectin-1 and -3. These observations suggest that at least two of the ten known galectins may be involved in anchoring Ras to the cell membrane.

5. Functional Relationships Between H-Ras (12V) and Galectin-1

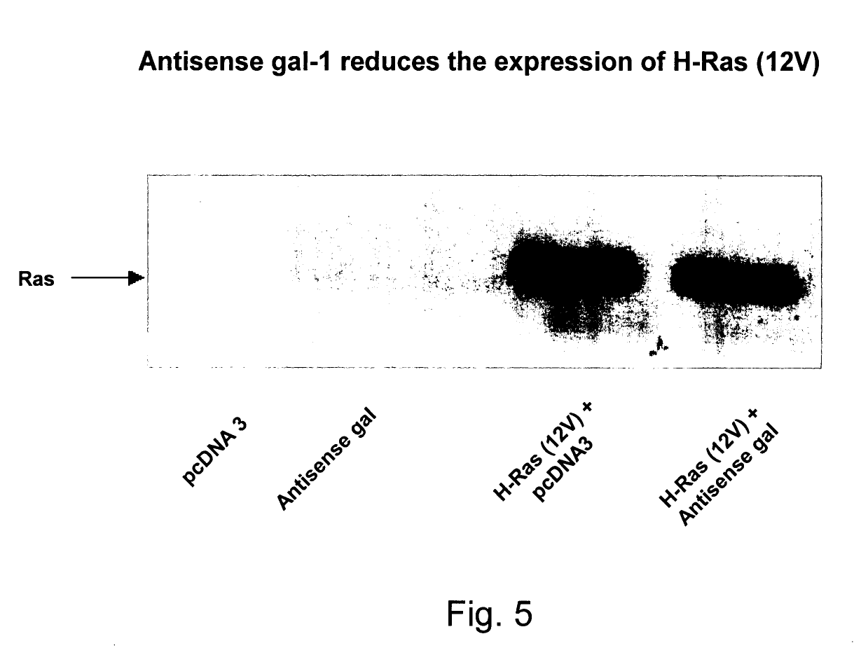

cDNA encoding Rat galectin-1 was obtained by RT-PCR using EJ cell RNA as a template as described in Clerch, et al. (1988). The cDNA was inserted into pcDNA either in the sense (pcDNA-gal-1) or anti-sense orientation. Transient transfection of the sense pcDNA-gal-1 into COS-7 and 293T cells resulted in a marked increase of galectin-1 as expected, due to an increase of its mRNA. To analyze the relationships between Ras and galectin-1, experiments were performed using galectin-1 antisense cDNA. Co-transfection of antisense pcDNA-gal-1 blocked of galectin-1 protein expression in COS-7 or 293T cells, thus validating the efficiency of gal-i antisense. COS-7 and 293T cells were then co-transfected with H-Ras (12V) cDNA in pcDNA or with H-Ras (12V) cDNA plus gal-1 antisense. As shown in FIG. 5, the gal-1 antisense caused a marked reduction in the concentration of H-Ras (12V). Similar experiments were performed with myr H-Ras (12V) and with N-Ras (13V). The results showed that gal-1 antisense had no effect on the concentration of these Ras isoforms, which are anchored by mechanisms that do not involve galectin-1. Thus, galectin-1 contributes rather specifically to the expression or the stabilization of H-Ras (12V). These findings also indicate that galectin-1 antisense had the same effect on H-Ras protein as FTS. This observation suggests that reduction of galectin-1 could have an anticancer effect on tumors driven by oncogenic H-Ras similar to that of FTS.

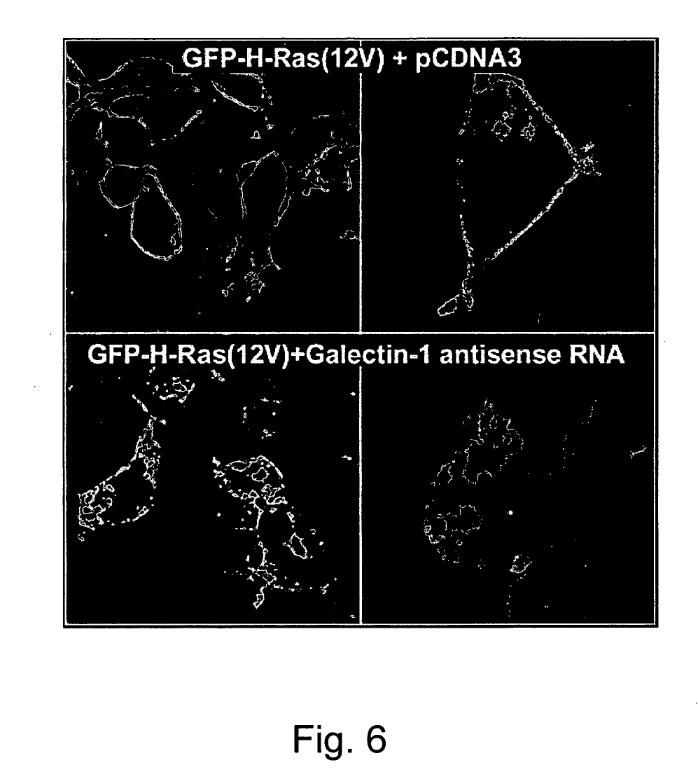

In a second set of experiments, green fluorescent protein (GFP)-labeled H-Ras (12V) was expressed alone or in combination with antisense gal-1 in COS-7 and in 293T cells. Confocal microscopy was used for localization of the GFP-H-Ras (12V). As in previous studies with GFP-K-Ras (12V), GFP-H-Ras (12V) localized on the cell membrane [Niv et al., 1999]. The co-transfection experiments clearly showed that the gal-1 antisense induced a strong reduction in GFP-H-Ras (12V) associated with cell membrane (FIG. 6). It is known from previous studies that GFP-Ras proteins, unlike their nonfused counterparts, are not readily degraded. Indeed, in this experiment we found that the gal-1 antisense caused a shift in the distribution of GFP-H-Ras (12V) from the cell membrane to cytoplasmic compartments (FIG. 6) and did not reduce the amount of GFP-H-Ras (12V) expressed by the cells. These results demonstrate that galectin-1 is an important protein for stabilization of H-Ras (12V) in a manner that permits its proper localization in the cell membrane.

### EXAMPLE 2

### Ras Anchor-Solid Phase Methods

Solid Phase Assays to Screen Novel Compounds that Interact with the Ras Anchors

Two independent solid-phase methods are used to assess the potency of new compounds as inhibitors of binding of Ras to its anchor protein(s). In method I, Ras protein is surface-immobilized onto microtiter plate wells and soluble biotin-labeled Ras anchor protein (e.g., galectin-1, galectin-3, galectin-7 or galectin-8) is then bound to the immobilized Ras in the absence (100% binding) or in the presence of various concentrations of a competitor. The apparent amount of galectin binding to the immobilized Ras is determined by streptavidin-peroxidase conjugate and o-phenylenediamine as a substrate. In method II, the Ras anchor protein is surface immobilized onto microtiter plate wells and soluble Ras is added in the absence (100% binding) and in the presence of various concentrations of the competitor. Pan mouse Ras antibody, secondary biotin-goat anti mouse IgG, streptavidin-peroxidase and a substrate o-phenylenediamine are added to determine the apparent amount of Ras binding. The reduction in OD₄₉₀ values in the presence of a competing compound as compared to the OD₄₉₀ recorded in its absence (100% binding) is indicative of the degree of inhibition of binding.

Method I: Assay with Immobilized Ras

Fully processed HA- tagged H-Ras (12V) and HA-tagged K-Ras (12V) are produced in insect cells and purified as detailed previously (Page, M. J. et al. 1989, J. Biol. Chem. 264, 19147-19154; Lowe, P.N. 1991, J. Biol. Chem. 266, 1672-1678). Biotin-galectin-1 and biotin- galectin -3 are prepared as detailed previously (Zeng, F.-J. and Gabius, H-.-J. 1993 in Gabius, H.-J. and Gabius, S. eds, Lectins and gliocobiology, Springer Pub. Co. Heidelber-New York, pp. 81-85; Ander', S. et al. 1997, Bioconjugate Chem. 8, 845-855).

Mouse anti-HA antibody (1 μg/ml, Jackson ImmunoResearch)) in sodium carbonate buffer pH 8.5/150 mM NaCl is added to each well for 30 min, the wells are then washed with 50 mM Tris HCl buffer pH 7.4, 0.1% octylglucoside, 0.1% bovine serum albumin (BSA), 1 mM MgCl₂ (buffer A). Ras protein (0.5 μg per well) in buffer A is then added to the wells for 1 h incubation at 25°. Following this Ras immobilization step and 3 times wash with buffer A, biotin-galectin -1 (for H-Ras assays) or biotin-galectin-3 (for K-Ras assays) is added in buffer A at a concentration of 5 μg/ml in the absence or in the presence of the competing compound. After a 2 h-incubation period at 25°, the wells are washed with 20 mM phosphate buffered saline pH 7.2/100 mM lactose (buffer B). The wells are then washed three times with 20 mM phosphate buffered saline pH 7.2 (buffer C) and streptavidin-peroxidase (0.5 μg/ml, Sigma) is added in buffer C for 1 h incubation at 25°. Following a 3 times wash with buffer C, o-phenylenediamine (1 mg/ml) and H₂O₂ μl/ml in buffer C are added. After 30 min-1 h incubation, the OD₄₉₀ values are determined with an automated ELISA reader.

Method II. Assay with Immobilized Galectin

Galectin-1 or galectin-3 are immobilized onto microtiter plates with asialofetuin prepared as detailed previously (Ander', S. et al. 1997, Bioconjugate Chem. 8, 845-855). Basically, 1 μg asialofetuin per well and 10 μg/ml of galectin-1 or 5 μg/ml of galectin-3 are used in buffer C. The wells are then washed with buffer A. Ras protein (1 μg per well in buffer A) is added in the absence and in the presence of the competing compound. Following a 2h-incubation period at 25°, the wells are washed 3 times in buffer A and mouse pan Ras antibody (1 μg, Calbiochem) is added in the same buffer for a 1 h incubation at 25°. Biotin conjugated goat anti-mouse antibody (1 μg/ml, Jackson ImmunoResearch) is then added, followed by streptaviden-peroxidase. The procedure then continues as detailed in Method I.

### INDUSTRIAL APPLICABILITY

The present invention provides methods and compositions disrupting and inhibiting underlying biochemical reactions in various disease states. It also provides methods for screening compounds for potential drugs that treat the diseases.

All patent and non-patent publications cited in this specification are indicative of the level of skill of those skilled in the art to which this invention pertains. All these publications and patent applications are herein incorporated by reference to the same extent as if each individual publication was specifically and individually indicated to be incorporated herein by reference.

### PUBLICATIONS

Aharonson, et al., Biochim. Biophys. Acta 1406(1):40-50 (1998)

Buss, et al., Science 243(4898):1600-1603 (1989)

Casey, et al., Proc. Natl. Acad. Sci. U.S. A. 86(21):8323-8327 (1989)

Cox, et al., Biochim. Biophys. Acta 1333(1):F51-71 (1997)

Cox, et al., Mol. Cell. Biol. 12(6):2606-2615 (1992)

Egozi, et al., Int. J. Cancer 80:911-918 (1999)

Elad, et al., BBA 1452:228-242 (1999)

Engelman, etal., J. Biol. Chem 272(26):16374-16381 (1997)

Gelb, et al., Curr Opin Chem Biol 2(1):40-48 (1998)

Haklai, et al., Biochemistry 37(5):1306-1314 (1998)

Hancock, et al., Cell 57(7):1167-1177 (1989)

Inouye, et al., J. Biol. Chem. 275(8):3737-3740 (2000)

Jansen, et al., Proc. Natl. Acad. Sci. USA 96:14019-14024 (1999)

Kloog, et al., Exp. Opin. Invest. Drugs 8(12):2121-2140 (1999)

Li, et al., J. Biol. Chem 271(46):29182-29190 (1996)

Marciano, et al., J. Med. Chem. 38(8):1267-1272 (1995)

Marciano, et al., Bioorg. Med. Chem. Lett 7(13):1709-1714 (1997)

Marom. et al., J. Biol. Chem. 270(38):22263-22270 (1995)

Mineo, et al., C, J. Biol. Chem. 271(20):11930-11935 (1996)

Mineo, et al., J. Biol. Chem. 272(16):10345-10348 (1997)

Niv, et al., J. Biol. Chem. 274(3):1606-1613 (1999)

Perillo, et al., J. Mol. Med. 76:402-412 (1998)

Schroeder, et al., Biochemistry 36(42):13102-13109 (1997)

Shahinian, et al., Biochemistry 34(11):3813-3822 (1995)

Siddiqui, et al., J. Biol. Chem. 273(6):3712-3717 (1998)

Song, et al., J. Biol. Chem. 271(16):9690-9697 (1996)

Weisz, et al., Oncogene 18:2579-2588 (1999)

Willumsen, Oncogene 13(9):1901-1909 (1996)

Wilson, et al., Biochem. J. 261(3):847-852 (1989)

Clerch, et al., Biochem. 27:692-699 (1988)

Gitt, et al., Biochem. 30(1):82-89 (1991)

Liu, et al., Proc. Natl. Acad. Sci. USA 84(19):6859-6863 (1987)

Pillai, Proc. Natl. Acad. Sci. USA 87(18):7324-7328 (1990)

Cherayil, et al., J. Exp. Med. 170(6):1959-1972 (1989)

Wagner, Nature Med. 1(11): 1116-1118 (1995)

Wagner, et al., Science 260:1510-1513 (1993)

1. A method for identifying a cell membrane anchor protein that binds a Ras protein, comprising:
preparing a first reaction mixture comprising the Ras protein, cell membranes or fragments thereof, and a Ras antagonist, and a second reaction mixture comprising the Ras protein and cell membranes or fragments thereof but not the Ras antagonist;
adding a cross-linking agent to the first and second reaction mixtures whereby cross-linked complexes between the Ras protein and other proteins are produced;
separating each of the cross-linked complexes individually;
identifying a complex formed in said second reaction mixture that is disrupted by the Ras antagonist present in said first reaction mixture;
separating thus-identified complex from other complexes; and
separating the Ras protein from the other protein in the separated complex.

2. The method of claim 1 wherein the antagonist is an inhibitor of a prenylated Ras protein.

3. The method of claim 1 wherein the antagonist is an inhibitor of a farnesylated Ras protein.

4. The method of claim 1 wherein the antagonist is S-trans, trans-farnesylthiosalicylic acid (FTS) or an analog thereof.

5. The method of claim 4 wherein the analog is 5-fluoro-FTS, 5-chloro-FTS, 4-chloro-FTS, 2-chloro-5-farnesylaminobenzoic acid, farnesyl thionicoatinic acid, S-farnesyl-methylthiosalicylic acid or 3-farnesylthio-cis-acrylic acid.

6. The method of claim 1 wherein the antagonist is an inhibitor of a non-prenylated Ras protein.

7. The method of claim 1 wherein the cell membranes are obtained from NIH fibroblasts transformed with oncogenic K-Ras 4B (12V), H-Ras (12V) or N-Ras (13V), 518A2/N-Ras melanoma cells, 607B melanoma cells, Panc-1 cells containing oncogenic K-Ras, EJ cells containing H-Ras (12V) or MC-MA-11 cells.

8. The method of claim 1 wherein the cross-linking agent is DSS.

9. The method of claim 1 wherein the cross-linking agent is DSP.

10. A method for identifying drug candidates that inhibit aberrant Ras activity, comprising:
preparing a reaction mixture containing a Ras protein, an anchor protein that binds the Ras protein and the drug candidate; and
determining effect of the drug candidate on interaction between the Ras protein and the anchor protein.

11. The method of claim 10 wherein said determining comprises measuring change in extent of dimerization of the Ras protein.

12. The method of claim 10 wherein said determining comprises measuring change in activation of Raf protein.

13. The method of claim 10 wherein said determining comprises measuring change in extent of binding of Raf protein to the Ras protein.

14. The method of claim 10 wherein said determining comprises measuring change in extent of binding between the Ras protein and the anchor protein.

15. The method of claim 14 wherein the reaction mixture further comprises a cross-linking agent.

16. The method of claim 10 wherein the Ras protein is immobilized on a matrix.

17. The method of claim 10 wherein the anchor protein is immobilized on a matrix.

18. The method of claim 10 wherein the anchor protein and the Ras protein are in solution.

19. The method of claim 10 wherein the anchor protein and/or the Ras protein are detectably labeled.

20. The method of claim 10 wherein anchor protein and/or the Ras protein are detectably labeled with a fluorescent protein.

21. The method of claim 20 wherein the fluorescent protein is green fluorescent protein or yellow fluorescent protein.

22. The method of claim 10 wherein the anchor protein comprises galectin-1.

23. The method of claim 10 wherein the anchor protein is galectin-3.

24. The method of claim 10 wherein the anchor protein is galectin-7.

25. The method of claim 10 wherein the anchor protein is galectin-8.

26. The method of claim 10 wherein the Ras protein and the anchor protein are provided in the form of living cells.

27. The method of claim 26 wherein said determining comprises measuring loss of the Ras protein from the anchor protein.

28. The method of claim 26 wherein said determining comprises observing intracellular movement of the Ras protein or the anchor protein.

29. A method disrupting aberrant Ras activity in vivo, comprising infusing into a patient exhibiting such aberrant Ras activity, a compound comprising an oligonucleotide molecule that binds mRNA of a Ras anchor protein and inhibits expression of the Ras anchor protein.

30. The method of claim 29 wherein the oligonucleotide binds galectin-1 mRNA.

31. The method of claim 29 wherein the oligonucleotide binds galectin-3 mRNA.

32. The method of claim 29 wherein the oligonucleotide binds galectin-7 mRNA.

33. The method of claim 29 wherein the oligonucleotide binds galectin-8 mRNA.

34. The method of claim 29 wherein the oligonucleotide contains at least one phosphorathioate-modified nucleotide.

35. The method of claim 29 wherein the oligonucleotide is administered to the patient via a liposome.

36. A method of determining efficacious dosages of a Ras antagonist that disrupts Ras-anchor protein binding, comprising:
contacting cells with the antagonist in vivo or in vitro;
collecting the cells following said contacting;
isolating cell membranes from the collected cells;
measuring decrease in anchor protein concentration per unit of cell membrane protein; and
correlating the decrease with dosage of the Ras antagonist.

37. An antisense compound that specifically binds a nucleic acid encoding galectin-1, galectin-3, galectin-7 or galectin-8, and which and which causes degradation of the nucleic acid.

38. A composition comprising the compound of claim 37 and a carrier.


SEQUENCE LISTING (33 sequences)

SEQ ID NO: 1 - protein, 135 aa, Rattus sp.
MACGLVASNLNLKPGECLKVRGELAPDAKSFVLNLGKDSNNLCLHFNPRFNAHGDANTIVCNSKDDGTWGTEQRETAFPFQPGSITEVCITFDQADLTIKLPDGHEFKFPNRLNMEAINYMAADGDFKIKCVAFE

SEQ ID NO: 2 - DNA, 408 bp, Rattus sp.
  CDS (1)..(405)
atggcctgtggtctggtcgccagcaacctgaatctcaaacctggggaatgtctcaaagttcggggagagctggccccggacgccaagagctttgtgttgaacctggggaaagacagcaacaacctgtgcctacacttcaacccccgcttcaacgcccacggagatgccaacaccattgtgtgtaacagcaaggacgatgggacctggggaacagaacaacgggagactgccttccctttccagcctgggagcatcacggaggtgtgcatcacctttgaccaggctgacctgaccatcaagctgccagacgggcatgaattcaaattccccaaccgcctcaacatggaggccatcaactacatggcggcggatggtgacttcaagattaagtgtgtggcctttgagtga

SEQ ID NO: 3 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 4 - DNA, 408 bp, Homo sapiens
  CDS (1)..(405)
atggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactga

SEQ ID NO: 5 - protein, 135 aa, Mus sp.
MACGLVASNLNLKPGECLKVRGEVASDAKSFVLNLGKDSNNLCLHFNPRFNAHGDANTIVCNTKEDGTWGTEHREPAFPFQPGSITEVCITFDQADLTIKLPDGHEFKFPNRLNMEAINYMAADGDFKIKCVAFE

SEQ ID NO: 6 - protein, 250 aa, Homo sapiens [sequence omitted]

SEQ ID NO: 7 - DNA, 753 bp, Homo sapiens
  CDS (1)..(750)
atggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccctggagcacctggagcttatcccggagcacctgcacctggagtctacccagggccacccagcggccctggggcctacccatcttctggacagccaagtgccaccggagcctaccctgccactggcccctatggcgcccctgctgggccactgattgtgccttataacctgcctttgcctgggggagtggtgcctcgcatgctgataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataa

SEQ ID NO: 8 - protein, 250 aa, Homo sapiens [sequence omitted]

SEQ ID NO: 9 - DNA, 753 bp, Homo sapiens; identical to SEQ ID NO: 7
  CDS (1)..(750)

SEQ ID NO: 10 - protein, 250 aa, Homo sapiens [sequence omitted]

SEQ ID NO: 11 - protein, 250 aa, Homo sapiens
MADNFSLHDALSGSGNPNPQGWPGAWGNQPAGAGGYPGASYPGAYPGQAPPGAYPGQAPPGAYHGAPGAYPGAPAPGVYPGPPSGPGAYPSSGQPSAPGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 12 - protein, 250 aa, Homo sapiens [sequence omitted]

SEQ ID NO: 13 - protein, 262 aa, Rattus sp.
MADGFSLNDALAGSGNPNPQGWPGAWGNQPGAGGYPGASYPGAYPGQAPPGGYPGQAPPSAYPGPTGPSAYPGPTAPGAYPGPTAPGAFPGQPGGPGAYPSAPGAYPSAPGAYPATGPFGAPTGPLTVPYDMPLPGGVMPRMLITIIGTVKPNANSITLNFKKGNDIAFHFNPRFNENNRRVIVCNTKQDNNWGREERQSAFPFESGKPFKIQVLVEADHFKVAVNDVHLLQYNHRMKNLREISQLGIIGDITLTSASHAMI

SEQ ID NO: 14 - protein, 263 aa, Mus sp.
MADSFSLNDALAGSGNPNPQGYPGAWGNQPGAGGYPGAAYPGAYPGQAPPGAYPGQAPPGAYPGQAPPSAYPGPTAPGAYPGPTAPGAYPGQPAPGAFPGQPGAPGAYPQCSGGYPAAGPGVPAGPLTVPYDLPLPGGVMPRMLITIMGTVKPNANRIVLDFRRGNDVAFHFNPRFNENNRRVIVCNTKQDNNWGKEERQSAFPFESGKPFKIQVLVEADHFKVAVNDAHLLQYNHRMKNLREISQLGISGDITLTSANHAMI

SEQ ID NO: 15 - protein, 136 aa, Homo sapiens
MSNVPHKSSLPEGIRPGTVLRIRGLVPPNASRFHVNLLCGEEQGSDAALHFNPRLDTSEVVFNSKEQGSWGREERGPGVPFQRGQPFEVLIIASDDGFKAVVGDAQYHHFRHRLPLARVRLVEVGGDVQLDSVRIF

SEQ ID NO: 16 - DNA, 411 bp, Homo sapiens
  CDS (1)..(408)
atgtccaacgtcccccacaagtcctcgctgcccgagggcatccgccctggcacggtgctgagaattcgcggcttggttcctcccaatgccagcaggttccatgtaaacctgctgtgcggggaggagcagggctccgatgccgccctgcatttcaacccccggctggacacgtcggaggtggtcttcaacagcaaggagcaaggctcctggggccgcgaggagcgcgggccgggcgttcctttccagcgcgggcagcccttcgaggtgctcatcatcgcgtcagacgacggcttcaaggccgtggttggggacgcccagtaccaccacttccgccaccgcctgccgctggcgcgcgtgcgcctggtggaggtgggcggggacgtgcagctggactccgtgaggatcttctga

SEQ ID NO: 17 - protein, 300 aa, Homo sapiens [sequence omitted]

SEQ ID NO: 18 - DNA, 903 bp, Homo sapiens
  CDS (1)..(900)
atgatgttgtccttaaacaacctacagaatatcatctatagcccggtaatcccgtatgttggcaccattcccgatcagctggatcctggaactttgattgtgatatgtgggcatgttcctagtgacgcagacagattccaggtggatctgcagaatggcagcagtgtgaaacctcgagccgatgtggcctttcatttcaatcctcgtttcaaaagggccggctgcattgtttgcaatactttgataaatgaaaaatggggacgggaagagatcacctatgacacgcctttcaaaagagaaaagtcttttgagatcgtgattatggtgctaaaggacaaattccaggttccaaagtctggcacgccccagcttcctagtaatagaggaggagacatttctaaaatcgcacccagaactgtctacaccaagagcaaagattcgactgtcaatcacactttgacttgcaccaaaataccacctacgaactatgtgtcgaagatcctgccattcgctgcaaggttgaacacccccatgggccctggcggcactgtcgtcgttaaaggagaagtgaatgcaaatgccaaaagctttaatgttgacctactagcaggaaaatcaaagcatattgctctacacttgaacccacgcctgaatattaaagcatttgtaagaaattcttttcttcaggagtcctggggagaagaagagagaaatattacctctttcccatttagtcctgggatgtactttgagatgataatttattgtgatgttagagaattcaaggttgcagtaaatggcgtacacagcctggagtacaaacacagatttaaagagctcagcagtattgacacgctggaaattaatggagacatccacttactggaagtaaggagctggtag

SEQ ID NO: 19 - DNA, 408 bp, Homo sapiens
tcagtcaaaggccacacatttgatcttgaagtcaccgtcagctgccatgtagttgatggcctccaggttgaggcggttggggaacttgaattcgtatccatctggcagcttgacggtcaggttggcctggtcgaaggtgatgcacacctctgcaacacttccaggctggaagggaaagacagcctcccgctgctcggtcccccaggccccgccgtccttgctgttgcacacgatggtgttggcgtcgccgtgggcgttgaagcgagggttgaagtgcaggcacaggttgttgctgtctttgcccaggttcagcacgaagctcttagcgtcaggagccacctcgcctcgcactcgaaggcactctccaggtttgagattcaggttgctggcgaccagaccacaagccat

SEQ ID NO: 20 - DNA, 25 bp, Homo sapiens
  antisense oligonucleotide
aagtcaccgtcagctgccatgtagt

SEQ ID NO: 21 - DNA, 23 bp, Homo sapiens
  antisense oligonucleotide
gatgcacacctctgcaacacttc

SEQ ID NO: 22 - DNA, 24 bp, Homo sapiens
  antisense oligonucleotide
tcagcacgaagctcttagcgtcag

SEQ ID NO: 23 - DNA, 22 bp, Homo sapiens
  antisense oligonucleotide
gcactcgaaggcactctccagg

SEQ ID NO: 24 - DNA, 22 bp, Homo sapiens
  antisense oligonucleotide
ggttgctggcgaccagaccaca

SEQ ID NO: 25 - DNA, 751 bp, Homo sapiens
ttatatcatggtatatgaagcactggtgaggtctatgtcaccagaaattcccagtttgctgatttcattgagttttttaacccgatgattgtactgcaacagtgagcatcattcactgcaaccttgaagtggtcaggttcaaccagtacttgtattttgaatggtttcccactttcaaatgggaaaaccgactgtctttcttcccttccccagttattatccagctttgtattgcaaacaatgactctcctgttgttctcattgaagcgtgggttaaagtggaaggcaacatcattccctctttggaaatctaaagcaattctgtttgcattgggcttcaccgtgcccagaattgttatcagcatgcgaggcaccactcccccaggcaaaggcaggttataaggcacaatcagtggcccagcaggggcgccataggggccagtggcagggtaggctccgggggcacttggctgtccagaagatgggtaggccccagggccgctgggtggccctgggtagactccaggtgcggtgctccgggataagctccaggtgctccatggtaggcgcctggaggtgcctgtccaggataagcccctgggggtgcctgcccggggtaggccccaggataggaagcccctgggtagccccctgccccagcaggctggttcccccatgcgccaggccatccttgagggtttgggtttccagacccagataacgcatcatggagcgaaaaattgtctgccat

SEQ ID NO: 26 - DNA, 22 bp, Homo sapiens
  antisense oligonucleotide
tatatgaagcactggtgaggtc

SEQ ID NO: 27 - DNA, 24 bp, Homo sapiens
  antisense oligonucleotide
gaagcgtgggttaaagtggaaggc

SEQ ID NO: 28 - DNA, 30 bp, Homo sapiens
  antisense oligonucleotide
ttgttatcagcatgcgaggcaccactcccc

SEQ ID NO: 29 - DNA, 21 bp, Homo sapiens
  antisense oligonucleotide
cacttggctgtccagaagatg

SEQ ID NO: 30 - DNA, 26 bp, Homo sapiens
  antisense oligonucleotide
gataagctccaggtgctccatggtag

SEQ ID NO: 31 - DNA, 20 bp, Homo sapiens
  antisense oligonucleotide
tccagacccagataacgcat

SEQ ID NO: 32 - DNA, 19 bp, Homo sapiens
  antisense oligonucleotide
tgtgggggacgttggacat

SEQ ID NO: 33 - DNA, 19 bp, Homo sapiens
  antisense oligonucleotide
tgtttaaggacaacatcat